(19) United States
(12) Patent Application Publication
(10) Pub. No.: US 2025/0382664 A1
(43) Pub. Date: Dec. 18, 2025

(54) IMPROVED DETECTION OF LAMP AMPLIFICATION PRODUCTS

(71) Applicant: Roche Molecular Systems, Inc., Pleasanton, CA (US)

(72) Inventor: Randall Saiki, Alameda, CA (US)

(21) Appl. No.: 18/723,774

(22) PCT Filed: Dec. 22, 2022
(86) PCT No.: PCT/EP2022/087400

§ 371 Date: Jun. 24, 2024

Related U.S. Application Data
(60) Provisional application No. 63/293,726, filed on Dec. 24, 2021.

(51) Int. Cl. C12Q 1/6844; C12Q 1/6818

(52) U.S. Cl. CPC C12Q 1/6844; C12Q 1/6818

(57) ABSTRACT

The present invention relates to methods to improve detection of amplification products from Loop-mediated Amplification (LAMP) reactions by the use of nucleotide analogs that form duplex structures that nucleic acid polymerase enzymes do not recognize efficiently.

### FIELD OF THE INVENTION

The present invention relates to the field of nucleic acid amplification and detection, in particular isothermal nucleic acid amplification and the detection of the amplification products.

### BACKGROUND OF THE INVENTION

Loop-mediated isothermal amplification (LAMP) is a unique isothermal amplification method having a number of advantages (See, e.g., U.S. Pat. No. 6,410,278 B1 and U.S. Pat. No. 7,374,913 B2). First, it is an isothermal nucleic acid amplification method, which eliminates the need for a specific device to control the temperature cycling, such as those needed for PCR, and thus it is ideally suited for point-of-care testing. Second, LAMP can generate a large amount of amplification products within a short period of time with high specificity. These two advantages enable relatively easy detection and a potential wide application in point-of-care testing. Another advantage is that, compared with other isothermal amplification methods, LAMP only requires one enzyme, a strand-displacing DNA polymerase, making the system easier to handle.

In addition to the DNA polymerase, LAMP uses 4 core primers (FIP, BTP, F3, and B3) recognizing 6 distinct sequence regions on the target (FIG. 1), with two primers containing sequence (F1C, B1C) that results in loop structures which facilitate exponential amplification (Notomi, et al., Nucleic Acids Res., 28:E63 (2000)). The use of multiple target sequence regions confers a high degree of specificity to the reaction. Two additional primers, termed loop primers, can be added to increase reaction speed, resulting in 6 total primers used per target sequence (Nagamine, et al., Mol. Cel. Probes, 16:223-9 (2002)). The LAMP reaction rapidly generates amplification products as multimers of the target region in various sizes, and is substantial in total DNA synthesis (>10 g, >50×PCR yield) (Notomi, et al. (2000); Nagamine, et al., Clin. Chem., 47:1742-3 (2001)).

Measurement of LAMP amplification product may be performed using fluorescence detection of double-stranded DNA, but these methods detect total DNA amplification in a reaction and are thus limited to detection of a single target. In order to utilize LAMP to detect multiple targets in a single sample, Tanner et al. (BioTechniques 53:81-89, 2012, and U.S. Pat. No. 9,074,249, each incorporated by reference herein in its entirety) developed a simple and clever modification called DARQ (Detection of Amplification by Release of Quenching) LAMP that allowed for the incorporation of fluorescent probes. In addition to improving sensitivity, fluorescent detection also permitted multiplexing of individually resolvable targets. In DARQ, the standard FIP primer is modified with a 5′ quencher and annealed to an F1c-complementary detection probe, Fd (see FIG. 2, top). To test DARQ LAMP detection, four sets of LAMP primers were designed with Q-FIP and accompanying Fd probes, each with a different fluorophore and quencher pair. The fluorescent probe used by Tanner forms a quenched duplex with one of the LAMP primers. Subsequent displacement of this probe by the strand-displacing Bst 2.0 DNA polymerase (New England Biolabs) during amplification allows fluorescent signal to accumulate. FIG. 2 depicts a graphical representation of the DARQ LAMP method.

One limitation of the DARQ LAMP method is the observed inhibition of amplification at high concentrations of the quenched-FIP primer (Q-FIP): Fd probe duplex. Although this inhibition could be significantly reduced through the use of equimolar standard FIP primer and Q-FIP:Fd duplex, fluorescence signal was also reduced by this process, which sometimes came down to undetectable levels. A method to perform DARQ LAMP without reduction of both amplification efficiency and signal generation would be highly desirable and increase the utility of the DARQ LAMP method for molecular diagnostics

### SUMMARY OF THE INVENTION

The present invention relates to an improved method to perform a DARQ LAMP assay by using an Fd detection probe that contains nucleotides that have the 2′ position of the ribose ring substituted with an O-methylated (OMe) moiety to form duplex structures that are not recognized efficiently by a DNA polymerase enzyme. Therefore, in one aspect, the present invention relates to a method for determining the presence or amount of a target nucleic acid molecule in a sample using loop mediated isothermal amplification (LAMP), the method comprising: (a) combining a LAMP reaction mixture, a DNA polymerase with strand displacement properties, a sample containing the target nucleic acid molecule; wherein the LAMP reaction mixture comprises: (i) a LAMP primer set of 4 to 6 primers, comprising two inner primers (FIP and BIP), two outer primers (F3 and B3), and optionally one or two loop primers (Loop F and/or Loop B); wherein the FIP primer comprises a primer sequence (F2) that is complementary to a sequence in the target nucleic acid molecule, and a tail sequence (F1c) that is not complementary to a sequence in the target nucleic acid molecule and that is 5′ of the primer sequence, and wherein the tail sequence is labeled with a quencher molecule; and (ii) a detection probe (Fd) capable of hybridizing to the tail sequence of the FIP primer to form a duplex, wherein the detection probe is labeled with a fluorescent molecule that is quenched by the quencher molecule when the detection probe is hybridized to the tail sequence, and wherein more than 80% of the nucleotides in the detection probe are substituted in the 2′ position of its ribose ring with a methoxy group (OMe substituted); b) amplifying the target nucleic acid molecule by LAMP under suitable assay conditions that allow generation of the target amplicons by the LAMP reaction and multiple cycles of displacing the detection probe from the tail sequence, thereby releasing the quenching of the fluorescent molecule to generate a detectable signal; and c) detecting and optionally quantifying the signal from the detection probe, and therefrom determining the presence or amount of the target nucleic acid molecule in the sample. In one embodiment, more than 90% of the nucleotides in the detection probe are OMe substituted. In another embodiment, all of the nucleotides in the detection probe are OMe substituted. In certain embodiments, the DNA polymerase with strand displacement properties is Bst polymerase. In another embodiment, the concentration of the detection probe is no more than 200 nM. In a further embodiment, the sample contains multiple target nucleic acid molecules, wherein the LAMP reaction mixture is a multiplex reaction mixture capable of amplifying and detecting multiple target nucleic acid molecules. In yet another embodiment, the multiplex reaction mixture comprises multiple detection probes, each probe being labeled with a different fluorescent molecule.

The details of one or more embodiments of the invention are set forth in the accompanying drawings and the description below. Other features, objects, and advantages of the invention will be apparent from the drawings and detailed description, and from the claims.

### BRIEF DESCRIPTION OF THE DRAWINGS

FIG. 1 is a graphical description of the basic LAMP reaction.

FIG. 2 is a graphical description of the DARQ LAMP reaction.

FIG. 3 shows the growth curves of the DARQ LAMP singleplex assays as described in Example 1.

FIG. 4 shows the growth curves of the DARQ LAMP multiplex assay as described in Example 2.

### DETAILED DESCRIPTION OF THE INVENTION

### Definitions

The term “sample” as used herein includes a specimen or culture (e.g., microbiological cultures) that includes nucleic acids. The term “sample” is also meant to include both biological and environmental samples. A sample may include a specimen of synthetic origin. Biological samples include whole blood, serum, plasma, umbilical cord blood, chorionic villi, amniotic fluid, cerebrospinal fluid, spinal fluid, lavage fluid (e.g., bronchioalveolar, gastric, peritoneal, ductal, ear, arthroscopic), biopsy sample, urine, feces, sputum, saliva, nasal mucous, prostate fluid, semen, lymphatic fluid, bile, tears, sweat, breast milk, breast fluid, embryonic cells and fetal cells. In a preferred embodiment, the biological sample is blood, and more preferably plasma. As used herein, the term “blood” encompasses whole blood or any fractions of blood, such as serum and plasma as conventionally defined. Blood plasma refers to the fraction of whole blood resulting from centrifugation of blood treated with anticoagulants. Blood serum refers to the watery portion of fluid remaining after a blood sample has coagulated. Environmental samples include environmental material such as surface matter, soil, water and industrial samples, as well as samples obtained from food and dairy processing instruments, apparatus, equipment, utensils, disposable and non-disposable items. These examples are not to be construed as limiting the sample types applicable to the present invention.

The terms “target” or “target nucleic acid” as used herein are intended to mean any molecule whose presence is to be detected or measured or whose function, interactions or properties are to be studied. Therefore, a target includes essentially any molecule for which a detectable probe (e.g., oligonucleotide probe) or assay exists, or can be produced by one skilled in the art. For example, a target may be a biomolecule, such as a nucleic acid molecule, a polypeptide, a lipid, or a carbohydrate, that is capable of binding with or otherwise coming in contact with a detectable probe (e.g., an antibody), wherein the detectable probe also comprises nucleic acids capable of being detected by methods of the invention. As used herein, “detectable probe” refers to any molecule or agent capable of hybridizing or annealing to a target biomolecule of interest and allows for the specific detection of the target biomolecule as described herein. In one aspect of the invention, the target is a nucleic acid, and the detectable probe is an oligonucleotide. The terms “nucleic acid” and “nucleic acid molecule” may be used interchangeably throughout the disclosure. The terms refer to oligonucleotides, oligos, polynucleotides, deoxyribonucleotide (DNA), genomic DNA, mitochondrial DNA (mtDNA), complementary DNA (cDNA), bacterial DNA, viral DNA, viral RNA, RNA, message RNA (mRNA), transfer RNA (tRNA), ribosomal RNA (rRNA), siRNA, catalytic RNA, clones, plasmids, M13, P1, cosmid, bacteria artificial chromosome (BAC), yeast artificial chromosome (YAC), amplified nucleic acid, amplicon, PCR product and other types of amplified nucleic acid, RNA/DNA hybrids and polyamide nucleic acids (PNAs), all of which can be in either single- or double-stranded form, and unless otherwise limited, would encompass known analogs of natural nucleotides that can function in a similar manner as naturally occurring nucleotides and combinations and/or mixtures thereof. Thus, the term “nucleotides” refers to both naturally-occurring and modified/non-naturally-occurring nucleotides, including nucleoside tri, di, and monophosphates as well as monophosphate monomers present within polynucleic acid or oligonucleotide. A nucleotide may also be a ribo; 2′-deoxy; 2′,3′-deoxy as well as a vast array of other nucleotide mimics that are well-known in the art. Mimics include chain-terminating nucleotides, such as 3′-O-methyl, halogenated base or sugar substitutions; alternative sugar structures including non-sugar, alkyl ring structures; alternative bases including inosine; deaza-modified; chi, and psi, linker-modified; mass label-modified; phosphodiester modifications or replacements including phosphorothioate, methylphosphonate, boranophosphate, amide, ester, ether; and a basic or complete internucleotide replacements, including cleavage linkages such a photocleavable nitrophenyl moieties.

The presence or absence of a target can be measured quantitatively or qualitatively. Targets can come in a variety of different forms including, for example, simple or complex mixtures, or in substantially purified forms. For example, a target can be part of a sample that contains other components or can be the sole or major component of the sample. Therefore, a target can be a component of a whole cell or tissue, a cell or tissue extract, a fractionated lysate thereof or a substantially purified molecule. Also a target can have either a known or unknown sequence or structure.

The term “amplification reaction” refers to any in vitro means for multiplying the copies of a target sequence of nucleic acid.

“Amplifying” refers to a step of submitting a solution to conditions sufficient to allow for amplification. Components of an amplification reaction may include, but are not limited to, e.g., primers, a polynucleotide template, polymerase, nucleotides, dNTPs and the like. The term “amplifying” typically refers to an “exponential” increase in target nucleic acid. However, “amplifying” as used herein can also refer to linear increases in the numbers of a select target sequence of nucleic acid, but is different than a one-time, single primer extension step.

“Polymerase chain reaction” or “PCR” refers to a method whereby a specific segment or subsequence of a target double-stranded DNA, is amplified in a geometric progression. PCR is well known to those of skill in the art; see, e.g., U.S. Pat. Nos. 4,683,195 and 4,683,202; and PCR Protocols: A Guide to Methods and Applications, Innis et al., eds, 1990.

“Oligonucleotide” as used herein refers to linear oligomers of natural or modified nucleosidic monomers linked by phosphodiester bonds or analogs thereof. Oligonucleotides include deoxyribonucleosides, ribonucleosides, anomeric forms thereof, peptide nucleic acids (PNAs), and the like, capable of specifically binding to a target nucleic acid. Usually monomers are linked by phosphodiester bonds or analogs thereof to form oligonucleotides ranging in size from a few monomeric units, e.g., 3-4, to several tens of monomeric units, e.g., 40-60. Whenever an oligonucleotide is represented by a sequence of letters, such as “ATGCCTG,” it will be understood that the nucleotides are in 5′-3′ order from left to right and that “A” denotes deoxyadenosine, “C” denotes deoxycytidine, “G” denotes deoxyguanosine, “T” denotes deoxythymidine, and “U” denotes the ribonucleoside, uridine, unless otherwise noted. Usually oligonucleotides comprise the four natural deoxynucleotides; however, they may also comprise ribonucleosides or non-natural nucleotide analogs. Where an enzyme has specific oligonucleotide or polynucleotide substrate requirements for activity, e.g., single stranded DNA, RNA/DNA duplex, or the like, then selection of appropriate composition for the oligonucleotide or polynucleotide substrates is well within the knowledge of one of ordinary skill.

As used herein “oligonucleotide primer”, or simply “primer”, refers to a polynucleotide sequence that hybridizes to a sequence on a target nucleic acid template and facilitates the detection of an oligonucleotide probe. In amplification embodiments of the invention, an oligonucleotide primer serves as a point of initiation of nucleic acid synthesis. In non-amplification embodiments, an oligonucleotide primer may be used to create a structure that is capable of being cleaved by a cleavage agent. Primers can be of a variety of lengths and are often less than 50 nucleotides in length, for example 12-25 nucleotides, in length. The length and sequences of primers for use in PCR can be designed based on principles known to those of skill in the art.

The term “oligonucleotide probe” as used herein refers to a polynucleotide sequence capable of hybridizing or annealing to a target nucleic acid of interest and allows for the specific detection of the target nucleic acid.

A “mismatched nucleotide” or a “mismatch” refers to a nucleotide that is not complementary to the target sequence at that position or positions. An oligonucleotide probe may have at least one mismatch, but can also have 2, 3, 4, 5, 6 or 7 or more mismatched nucleotides.

The term “polymorphism” as used herein refers to an allelic variant. Polymorphisms can include single nucleotide polymorphisms (SNP's) as well as simple sequence length polymorphisms. A polymorphism can be due to one or more nucleotide substitutions at one allele in comparison to another allele or can be due to an insertion or deletion, duplication, inversion and other alterations known to the art.

The term “modification” as used herein refers to alterations of the oligonucleotide probe at the molecular level (e.g., base moiety, sugar moiety or phosphate backbone). Nucleoside modifications include, but are not limited to, the introduction of cleavage blockers or cleavage inducers, the introduction of minor groove binders, isotopic enrichment, isotopic depletion, the introduction of deuterium, and halogen modifications. Nucleoside modifications may also include moieties that increase the stringency of hybridization or increase the melting temperature of the oligonucleotide probe. For example, a nucleotide molecule may be modified with an extra bridge connecting the 2′ and 4′ carbons resulting in locked nucleic acid (LNA) nucleotide that is resistant to cleavage by a nuclease. The compositions of the tag portion of the oligonucleotide probe and of the capture oligonucleotide molecule are only restricted by their ability to form stable duplexes. These oligonucleotides can therefore comprise of DNA, L-DNA, RNA, L-RNA, LNA, L-LNA, PNA, BNA, L-BNA etc. (where the “L-XXX” refers to the L-enantiomer of the sugar unit of the nucleic acids) or any other known variations and modifications on the nucleotide bases, sugars, or phosphodiester backbones.

Other examples of nucleoside modifications include various 2′ substitutions such as halo, alkoxy and allyloxy groups that are introduced in the sugar moiety of oligonucleotides. Evidence has been presented that 2′-substituted-2′-deoxyadenosine polynucleotides resemble double-stranded RNA rather than DNA. Ikehara et al., (Nucleic Acids Res., 1978, 5, 3315) have shown that a 2′-fluro substituent in poly A, poly I, or poly C duplexed to its complement is significantly more stable than the ribonucleotide or deoxyribonucleotoide poly duplex as determined by standard melting assays. Inoue et al., (Nucleic Acids Res., 1987, 15, 6131) have described the synthesis of mixed oligonucleotide sequences containing 2′-OMe (0-Methyl) substituents on every nucleic nucleotide. The mixed 2′-OMe-substituted oligonucleotide hybridized to its RNA complement as strongly as the RNA-RNA duplex, which is significantly stronger than the same sequence RNA-DNA heteroduplex. Therefore, examples of substitutions at the 2′ position of the sugar include F, CN, CF3, OCF3, OMe, OCN, O-alkyl, S-alkyl, SMe, SO2Me, ONO2, NO2, NH3, NH2, NH-alkyl, OCH3=CH2 and OCCH.

The term “specific” or “specificity” in reference to the binding of one molecule to another molecule, such as a probe for a target polynucleotide, refers to the recognition, contact, and formation of a stable complex between the two molecules, together with substantially less recognition, contact, or complex formation of that molecule with other molecules. As used herein, the term “anneal” refers to the formation of a stable complex between two molecules.

A probe is “capable of annealing” to a nucleic acid sequence if at least one region of the probe shares substantial sequence identity with at least one region of the complement of the nucleic acid sequence. “Substantial sequence identity” is a sequence identity of at least about 80%, preferably at least about 85%, more preferably at least about 90%, 95% or 99%, and most preferably 100%. For the purpose of determining sequence identity of a DNA sequence and a RNA sequence, U and T often are considered the same nucleotide. For example, a probe comprising the sequence ATCAGC is capable of hybridizing to a target RNA sequence comprising the sequence GCUGAU.

A “nucleic acid polymerase” refers to an enzyme that catalyzes the incorporation of nucleotides into a nucleic acid. Exemplary nucleic acid polymerases include DNA polymerases, RNA polymerases, terminal transferases, reverse transcriptases, telomerases and the like.

### Fluorescence Resonance Energy Transfer (FRET)

FRET technology (see, for example, U.S. Pat. Nos. 4,996,143, 5,565,322, 5,849,489, and 6,162,603) is based on a concept that when a donor fluorescent moiety and a corresponding acceptor fluorescent moiety are positioned within a certain distance of each other, energy transfer takes place between the two fluorescent moieties that can be visualized or otherwise detected and/or quantitated. The donor typically transfers the energy to the acceptor when the donor is excited by light radiation with a suitable wavelength. The acceptor typically re-emits the transferred energy in the form of light radiation with a different wavelength. In certain systems, non-fluorescent energy can be transferred between donor and acceptor moieties, by way of biomolecules that include substantially non-fluorescent donor moieties (see, for example, U.S. Pat. No. 7,741,467).

In one example, an oligonucleotide probe can contain a donor fluorescent moiety and a corresponding quencher, which may or not be fluorescent, and which dissipates the transferred energy in a form other than light. When the probe is intact, energy transfer typically occurs between the two fluorescent moieties such that fluorescent emission from the donor fluorescent moiety is quenched. During an extension step of a polymerase chain reaction, a probe bound to an amplification product is cleaved by the 5′ to 3′ nuclease activity of, e.g., a Taq Polymerase such that the fluorescent emission of the donor fluorescent moiety is no longer quenched. Exemplary probes for this purpose are described in, e.g., U.S. Pat. Nos. 5,210,015, 5,994,056, and 6,171,785. Commonly used donor-acceptor pairs include the FAM-TAMRA pair. Commonly used quenchers are DABCYL and TAMRA. Commonly used dark quenchers include BlackHole Quenchers™ (BHQ), (Biosearch Technologies, Inc., Novato, Cal.), Iowa Black™ (Integrated DNA Tech., Inc., Coralville, Iowa), BlackBerry™ Quencher 650 (BBQ-650), (Berry & Assoc., Dexter, Mich.).

In another example, two oligonucleotide probes, each containing a fluorescent moiety, can hybridize to an amplification product at particular positions determined by the complementarity of the oligonucleotide probes to the target nucleic acid sequence. Upon hybridization of the oligonucleotide probes to the amplification product nucleic acid at the appropriate positions, a FRET signal is generated. Hybridization temperatures can range from about 35° C. to about 65° C. for about 10 sec to about 1 min.

Fluorescent analysis can be carried out using, for example, a photon counting epifluorescent microscope system (containing the appropriate dichroic mirror and filters for monitoring fluorescent emission at the particular range), a photon counting photomultiplier system, or a fluorimeter. Excitation to initiate energy transfer, or to allow direct detection of a fluorophore, can be carried out with an argon ion laser, a high intensity mercury (Hg) arc lamp, a fiber optic light source, or other high intensity light source appropriately filtered for excitation in the desired range.

As used herein with respect to donor and corresponding acceptor fluorescent moieties “corresponding” refers to an acceptor fluorescent moiety having an absorbance spectrum that overlaps the emission spectrum of the donor fluorescent moiety. The wavelength maximum of the emission spectrum of the acceptor fluorescent moiety should be at least 100 nm greater than the wavelength maximum of the excitation spectrum of the donor fluorescent moiety. Accordingly, efficient non-radiative energy transfer can be produced therebetween.

Fluorescent donor and corresponding acceptor moieties are generally chosen for (a) high efficiency Forster energy transfer; (b) a large final Stokes shift (>100 nm); (c) shift of the emission as far as possible into the red portion of the visible spectrum (>600 nm); and (d) shift of the emission to a higher wavelength than the Raman water fluorescent emission produced by excitation at the donor excitation wavelength. For example, a donor fluorescent moiety can be chosen that has its excitation maximum near a laser line (for example, Helium-Cadmium 442 nm or Argon 488 nm), a high extinction coefficient, a high quantum yield, and a good overlap of its fluorescent emission with the excitation spectrum of the corresponding acceptor fluorescent moiety. A corresponding acceptor fluorescent moiety can be chosen that has a high extinction coefficient, a high quantum yield, a good overlap of its excitation with the emission of the donor fluorescent moiety, and emission in the red part of the visible spectrum (>600 nm).

Representative donor fluorescent moieties that can be used with various acceptor fluorescent moieties in FRET technology include fluorescein, Lucifer Yellow, B-phycoerythrin, 9-acridineisothiocyanate, Lucifer Yellow VS, 4-acetamido-4′-isothio-cyanatostilbene-2,2′-disulfonic acid, 7-diethylamino-3-(4′-isothiocyanatophenyl)-4-methylcoumarin, succinimdyl 1-pyrenebutyrate, and 4-acetamido-4′-isothiocyanatostilbene-2,2′-disulfonic acid derivatives. Representative acceptor fluorescent moieties, depending upon the donor fluorescent moiety used, include LC Red 640, LC Red 705, Cy5, Cy5.5, Lissamine rhodamine B sulfonyl chloride, tetramethyl rhodamine isothiocyanate, rhodamine x isothiocyanate, erythrosine isothiocyanate, fluorescein, diethylenetriamine pentaacetate, or other chelates of Lanthanide ions (e.g., Europium, or Terbium). Donor and acceptor fluorescent moieties can be obtained, for example, from Molecular Probes (Junction City, Oreg.) or Sigma Chemical Co. (St. Louis, Mo.).

The donor and acceptor fluorescent moieties can be attached to the appropriate probe oligonucleotide via a linker arm. The length of each linker arm is important, as the linker arms will affect the distance between the donor and acceptor fluorescent moieties. The length of a linker arm can be the distance in Angstroms (Å) from the nucleotide base to the fluorescent moiety. In general, a linker arm is from about 10 Å to about 25 Å. The linker arm may be of the kind described in WO 84/03285. WO 84/03285 also discloses methods for attaching linker arms to a particular nucleotide base, and also for attaching fluorescent moieties to a linker arm.

An acceptor fluorescent moiety, such as an LC Red 640, can be combined with an oligonucleotide which contains an amino linker (e.g., C6-amino phosphoramidites available from ABI (Foster City, Calif) or Glen Research (Sterling, VA)) to produce, for example, LC Red 640-labeled oligonucleotide. Frequently used linkers to couple a donor fluorescent moiety such as fluorescein to an oligonucleotide include thiourea linkers (FITC-derived, for example, fluorescein-CPG's from Glen Research or ChemGene (Ashland, Mass.)), amide-linkers (fluorescein-NHS-ester-derived, such as CX-fluorescein-CPG from BioGenex (San Ramon, Calif)), or 3′-amino-CPGs that require coupling of a fluorescein-NHS-ester after oligonucleotide synthesis.

### LAMP and DARQ LAMP Methodologies

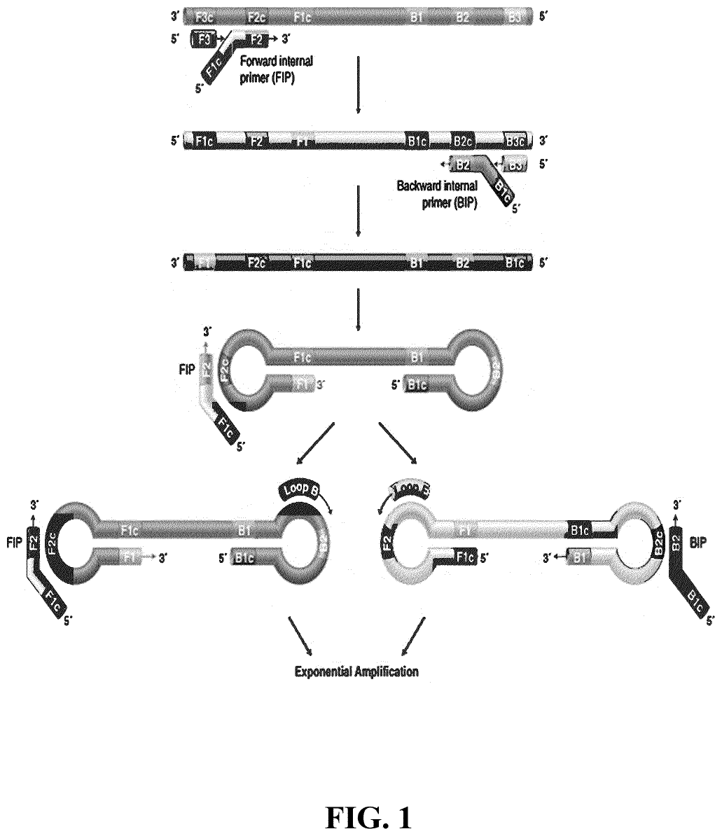

“LAMP” or “Loop Mediated Isothermal Amplification” refers to an isothermal amplification method, i.e. a method that is performed at an essential constant temperature without the need for a thermocycler. In LAMP, the target sequence is typically amplified at 60° C. to 65° C. using either two or three sets of primers (i.e., 4 to 6 primers) and a polymerase with high strand displacement activity in addition to a replication activity. DNA polymerase with strand displacement activity/properties is known to those skilled in the art as an ability of the polymerase to displace the downstream DNA strand encountered during synthesis along the target strand. Typically, four different primers are used to identify six distinct regions on the target gene, which adds highly to the specificity (FIG. 1). An additional “loop primer” or pair of “loop primers” can further accelerate the reaction. Due to the specific nature of the action of these primers, the amount of DNA produced in LAMP is considerably higher than PCR based amplification. The LAMP method is described in U.S. Pat. Nos. 6,410,278 B1 and 7,374,913 B2. Generally, the method uses two inner primers (forward inner primer=FIP and backward inner primer=BIP), two outer primers (F3 and B3), and optionally one or two, preferably two, loop primers (loop forward=LF and/or loop backward=LB). If two loop primers are used, one is preferably a loop forward primer and the other a loop backward primer. The inner primers comprise a target complementary region (typically referred to as F2 and B2) that facilitates hybridization and 5′ thereto a sequence that is identical to a sequence in the target nucleic acid located upstream (5′) relative to the sequence of the target bound by the target complementary region of the inner primer (typically referred to as F1c and B1c). Elongation of the inner primer by the polymerase thus creates a sequence comprising regions of self-complementarity in that the target-identical sequence on the 5′ end of the inner primer (B1c) can, after elongation, bind to the synthesized sequence downstream of the target-complementary region of the inner primer (referred to as B1) and act as a primer for further extension. The outer primers bind to a target region in the target nucleic acid that lies downstream (i.e. 3′) to the target region bound by the inner primers (referred to as F3c and B3c) and thus are responsible for the displacement of the elongated inner primer sequences from the template strand. The elongated inner primers are recognized and hybridized by the other primer of the inner primer pair and thus the dumbbell structured starting amplicons are generated. The dumbbell structures are then used for the following amplification, with the amplicons taking the form of concatemers. The principles of LAMP are for example disclosed by Eiken Chemical Co., Ltd. in the publications of Nagamine et al. (Mol. Cell. Probes (2002) 16:223-229) and Notomi et al. (Nucleic Acids Res. (2000), 28 (12): e63) and the architecture of LAMP targets and primers is also schematically shown in FIG. 1.

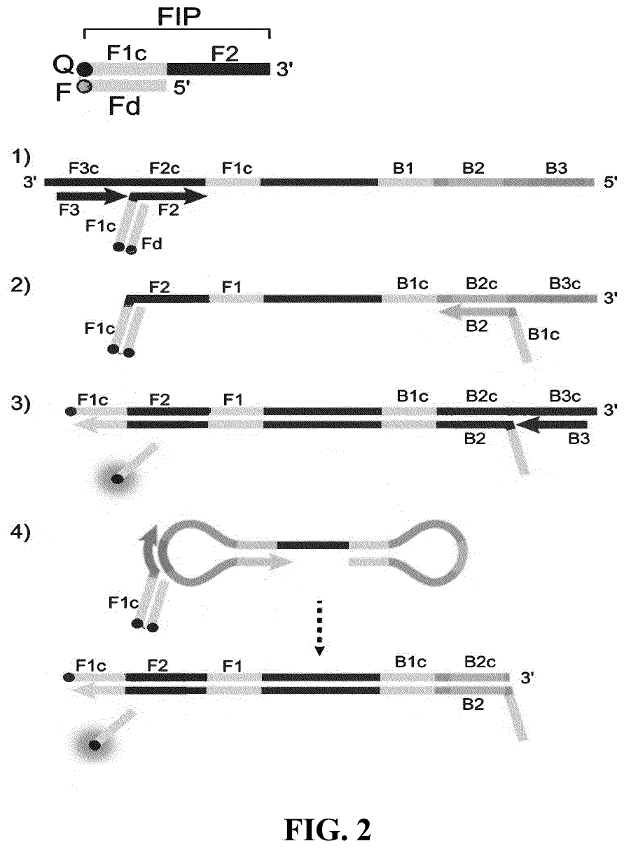

DARQ (Detection of Amplification by Release of Quenching) LAMP is a modification of the LAMP method that enables the fluorescence detection of the LAMP amplification products in a multiplex reaction. The LAMP forward and back interior primers (FIP and BIP) contain 5′ flaps (FIG. 2, F1c sequence) that, upon synthesis and displacement, will anneal to a complementary downstream region (F1). This region was chosen for the development of detection probes, as it is inherent to LAMP and contains sequence that is specific to each target, precluding any need for probe sequence optimization. LAMP also requires a strand displacing DNA polymerase (typically Bst DNA polymerase, large fragment), a component utilized for detection through strand displacement. Using previously designed LAMP primers as a basis, the FIP modified at the 5′ end was synthesized with a dark quencher. For probe creation (Fd), oligonucleotides complementary to the flap region (F1c) was annealed with a fluorophore spectrally overlapping with the dark quencher of the FIP (FIG. 2, Q-FIP:Fd duplex). This duplex primer maintains its function as a LAMP primer, but upon synthesis from the reverse direction the flap duplex is separated, resulting in detection of amplification by release of quenching (FIG. 2, Step 3).

However, it has been shown in DARQ LAMP that a high concentration(1.6 μM) of the Q-FIP:Fd duplex inhibits the LAMP amplification process and therefore, the concentration of the Fd probe needed to be reduced which, in turn, lowers the detectable fluorescence signal. One possible reason for the inhibition of amplification is the non-productive binding of DNA polymerase to the FIP:Fd duplex. In way to eliminate this binding of the polymerase to the duplex is to synthesize the Fd probe with nucleotide analogs such as L-DNA or 2′-O-methylated nucleotides), which form duplex structures that the polymerase enzyme does not recognize efficiently. The 2′-O-methyl-substituted (OMe) Fd probe will hybridize with normal bases in the F1c portion of the primer but polymerase should not bind to a OMe:DNA duplex.

Embodiments of the present invention will be further described in the following examples, which do not limit the scope of the invention described in the claims.

### EXAMPLES

### Example 1: Performance of Native DNA Vs OMe DARQ LAMP Probes

A DARQ LAMP experiment was performed according to the methods described in Tanner et al, BioTechniques 53:81-89, 2012, for amplification and detection of three of the target genes: Escherichia coli dnaE, Caenorhabditis elegans lec10, and human BRCA-1. Experimental conditions were as follows. Q-FIP:Fd duplexes were annealed by heating 50 μM Q-FIP and 50 μM Fd to 98° C. and slowly cooling the mixture to room temperature. LAMP reactions with Bst 2.0 DNA polymerase or Bst 2.0 WarmStart DNA polymerase (New England Biolabs, Ipswich, MA, USA) were performed in 1× Isothermal Amplification Buffer (New England Biolabs): 20 mM Tris-HCl (pH 8.8, 25° C.), 10 mM (NH₄)₂SO₄, 50 mM KCl, 2 mM MgSO₄, 0.1% Tween-20 supplemented to 8 mM MgSO₄, and 1.4 mM each of dATP, dCTP, dGTP, and dTTP. LAMP reactions contained: 1.6 μM FIP (or 0.8 μM FIP and 0.8 μM Q-FIP:Fd), 1.6 μM BIP, 0.2 μM F3 and B3, 0.4 μM LoopF and LoopB, and 0.64 U/μL Bst2.0 DNA polymerase, or Bst 2.0 WarmStart DNA polymerase. Both native and OMe substituted Fd probes were labeled with the BHQ-2 dark quencher while the FIP primers annealing to dnaE, lec10 and BRCA-1 were labeled with the fluorescent dyes, Coumarin (COU), FAM and HEX, respectively. Synthetic templates were added at 2,500 copies per reaction.

TABLE 1

| | | D-DNA | 2′-OMe |
|---|---|---|---|
| Target | Dye | (detection time) | (detection time) |
| dnaE | COU | 25.5 minutes | 10.5 minutes |
| lec10 | FAM | 20.1 minutes | 8.2 minutes |
| BRCA1 | HEX | 23.8 minutes | 14.5 minutes |

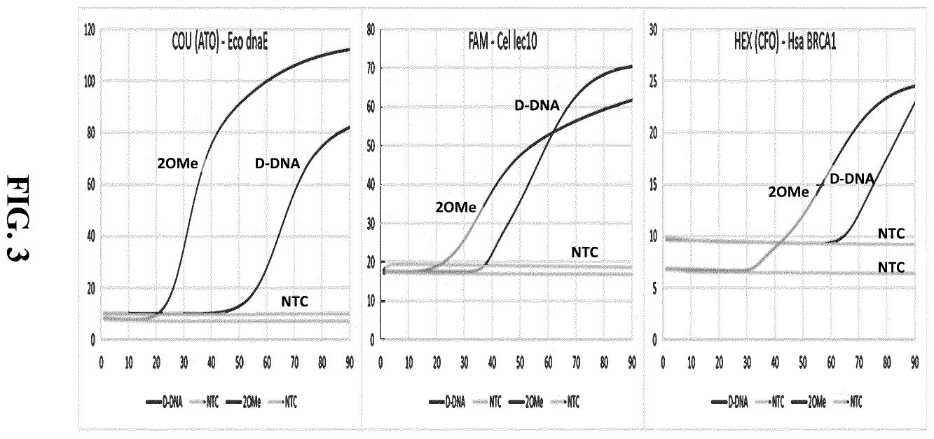

The results of the experiment are shown as detection times in TABLE 1 and as growth curves in FIG. 3. Compared to “native” D-DNA Fd probes, Fd probes with OMe substitutions exhibited detection times that were approximately 2× faster and also generated significantly stronger fluorescence signals.

### Example 2: Multiplexing of Five Targets with OMe DARQ LAMP

In order to demonstrate the performance of DARQ LAMP assays using 2′-OMe substituted Fd probes, a five-target multiplex assay was designed with the following four gene targets: SARS-CoV-2 N gene (SC2), FluA Hemagglutinin H1 gene (AH1), FluA Hemagglutinin H3 gene (AH3), FluB Neuraminidase gene (BNA), and one internal control (GIC). DARQ LAMP 5-plex reactions were performed with 400 nM each FIP/BIP, 100 nM each F3/B3, 400 nM each Loop F/Loop B, and 200 nM each Fd probe in 1× IsoAmp Buffer (New England Biolabs), 1.5 mM dATP, 1.5 mM dCTP, 1.5 mM dGTP, 1.5 mM dTTP, 6 mM MgSO4, 40 mM guanine hydrochloride, and 64 U Bst 2.0 Warm Start polymerase (New England Biolabs) in a 50 μL volume. Reactions were incubated at 65° C. for 60 min in at LightCycler 480 (Roche) set to collect fluorescence data—every 0.5 min. The following fluorescent dyes were used for each of the Fd probes: Coumarin for SC2, FAM for AH1, HEX for AH3, JA270 for BNA, and Cy5.5 for GIC. When present, single-stranded K562 human genomic DNA (Promega) was added at 100 ng/reaction.

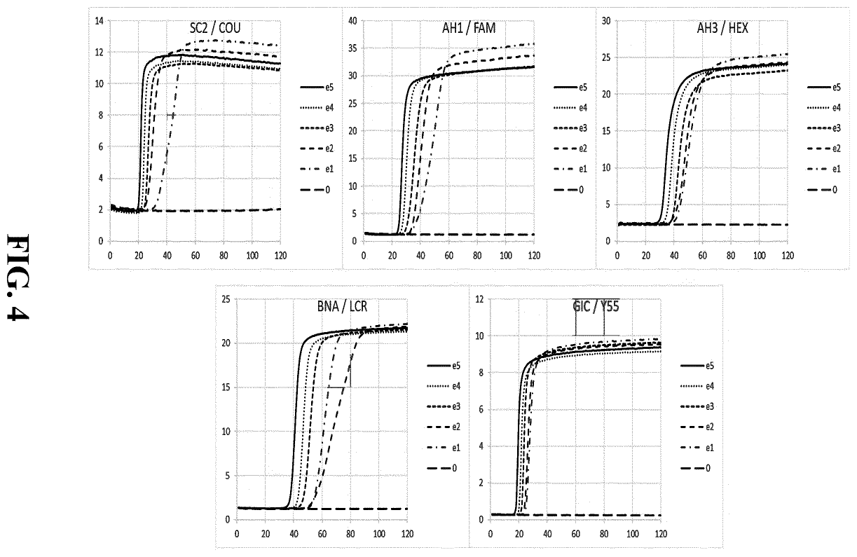

Performance of the OMe DARQ LAMP multiplex reaction was tested using single stranded synthetic templates at concentrations of 10, 100, 1000, 10000 and 100000 copies per reaction and the results and shown in TABLE 2 as detection times and in FIG. 4 as growth curves. Three of the templates (AH3, BNA, GIC) showed robust detection down to 10 copies per reaction whereas two of the templates (SC2, AH1) exhibited detection down to 100 copies per reaction.

TABLE 2

| | |
|---|---|
| Target | Detection Time (min) |

| cp/rxn | 100,000 | 10,000 | 1,000 | 100 | 10 |
|---|---|---|---|---|---|
| SC2 | 10.1 | 11.6 | 13.0 | 13.9 | ND |
| AH1 | 12.3 | 13.7 | 15.6 | 17.8 | ND |
| AH3 | 14.8 | 16.5 | 17.7 | 20.4 | 21.2 |
| BNA | 20.8 | 23.4 | 25.0 | 25.7 | 28.6 |
| GIC | 9.2 | 10.4 | 11.6 | 12.2 | 13.7 |

ND = Not Detected

While the foregoing invention has been described in some detail for purposes of clarity and understanding, it will be clear to one skilled in the art from a reading of this disclosure that various changes in form and detail can be made without departing from the true scope of the invention. For example, the methods described above can be used in various combinations. All publications, patents, patent applications, and/or other documents cited in this application are incorporated by reference in their entirety for all purposes to the same extent as if each individual publication, patent, patent application, and/or other document were individually indicated to be incorporated by reference for all purposes.

INFORMAL SEQUENCE LISTING

| | | |
|---|---|---|
| SEQ ID NO: 1 | TGGCTACTACCGAAGAGCT | SC2-N, F3 |
| SEQ ID NO: 2 | TGCAGCATTGTTAGCAGGA | SC2-N, B3 |
| | T | |
| SEQ ID NO: 3 | QTCTGGCCCAGTTCCTAGG | SC2-N, FIP, |
| | TAGTccagacgaattcgtg | BHQ2 |
| | gtgg | |
| SEQ ID NO: 4 | AGACGGCATCATATGGGTT | SC2-N, BIP |
| | GCAcgggtgccaatgtgat | |
| | ct | |
| SEQ ID NO: 5 | GGACTGAGATCTTTCATTT | SC2-N, Loop |
| | TACCGT | F |
| SEQ ID NO: 6 | ACTGAGGGAGCCTTGAATA | SC2-N, Loop |
| | CA | B |
| SEQ ID NO: 7 | ACUACCUAGGAACUGGGCC | SC2-N, Probe, |
| | AGAK | OMe, COU |
| SEQ ID NO: 8 | AGCAAGAAGTTCAAGCCG | FluA H1, F3 |
| SEQ ID NO: 9 | CGTGAACTGGTGTATCTGA | FluA H1, B3 |
| | A | |
| SEQ ID NO: 10 | QGGCTCTACTAGTGTCCAG | FluA H1, |
| | TAATAGTCCCCaaatagca | FIP + 4C, |
| | ataagacccaaagtg | IBRQ |
| SEQ ID NO: 11 | ATAACATTCGAAGCAACTG | FluA H1, BIP |
| | GAAATCtgataataccaga | |
| | tccagcatt | |
| SEQ ID NO: 12 | TCTCCCTTCTTGATCCC | FluA H1, |
| | | Loop F |
| SEQ ID NO: 13 | TAGTGGTACCGAGATATGC | FluA H1, |
| | A | Loop B |
| SEQ ID NO: 14 | GGGGACUAUUACUGGACAC | FluA H1, |
| | UAGUAGAGCCF | Probe + 4G |
| | | OMe, FAM |
| SEQ ID NO: 15 | GGGGTTACTTCAAAATACG | FluA H3, F3 |
| SEQ ID NO: 16 | GTTGCCAATTTCAGAGTG | FluA H3, B3 |
| SEQ ID NO: 17 | QGAGTGATGCATTCAGAAT | FluA H3, |
| | TGCATTTCCCCtgggaaaa | FIP + 4C, |
| | gctcaataatgaga | IBRQ |
| SEQ ID NO: 18 | AATGGAAGCATTCCCAATG | FluA H3, BIP |
| | ACAgcttaacatatctggg | |
| | acagg | |
| SEQ ID NO: 19 | CCAATGGGTGCATCTGA | FluA H3, |
| | | Loop F |
| SEQ ID NO: 20 | AACCATTCCAAAATGTAAA | FluA H3, |
| | C | Loop B |
| SEQ ID NO: 21 | GGGGAAAUGCAAUUCUGAA | FluA H3, |
| | UGCAUCACUCH | Probe + 4G |
| | | OMe, HEX |
| SEQ ID NO: 22 | CAGGAAGAGTAAAACATAC | FluB NA, F3 |
| | TGAGGA | |
| SEQ ID NO: 23 | GATTCGCAAGGCCCTGTT | FluB NA, B3 |
| SEQ ID NO: 24 | QAGGGTCTTTTTGCTGTGT | FluB NA, |
| | AACTGTTCCCCgcacatgc | FIP + 4C, |
| | ggatttgccag | IBRQ |
| SEQ ID NO: 25 | GTGGAGACTGATACAGCGG | FluB NA, BIP |
| | AAtgcttccatcatttggt | |
| | ctgg | |
| SEQ ID NO: 26 | CTACAGGCACATTCTATGG | FluB NA, |
| | TT | Loop F |
| SEQ ID NO: 27 | ATAAGATTGATGTGCACA | FluB NA, |
| | | Loop B |
| SEQ ID NO: 28 | GGGGAACAGUUACACAGCA | FluB NA, |
| | AAAAGACCCUJ | Probe + 4G |
| | | OMe, JA2 |
| SEQ ID NO: 29 | CGATTTTGGTTCCGGTCAC | GIC, F3 |
| | A | |
| SEQ ID NO: 30 | CGGGCCGCTTATACAGTAC | GIC, B3 |
| SEQ ID NO: 31 | CGCCCCGACTCACTTAATT | GIC, FIP |
| | GCTccgcgccatacatgtc | |
| | aa | |
| SEQ ID NO: 32 | QTGACTCCCACGTAAAAAG | GIC, BIP, |
| | CCCCcaatcgtcggttcgt | BHQ2 |
| | gcg | |
| SEQ ID NO: 33 | CGTCAGTTTTCTAGCGTTC | GIC, Loop F |
| | GCC | |
| SEQ ID NO: 34 | GCACCGTTACGAAGTATCA | GIC, Loop B |
| | AAACG | |
| SEQ ID NO: 35 | GGGGCUUUUUACGUGGGAG | GIC, Probe, |
| | UCAY | OMe, Y55 |

1. A method for determining the presence or amount of a target nucleic acid molecule in a sample using loop mediated isothermal amplification (LAMP), the method comprising:
(a) combining a LAMP reaction mixture, a DNA polymerase with strand displacement properties, and a sample containing the target nucleic acid molecule; wherein the LAMP reaction mixture comprises:
(i) a LAMP primer set of 4 to 6 primers, comprising two inner primers (FIP and BIP), two outer primers (F3 and B3), and optionally one or two loop primers (Loop F and/or Loop B); wherein the FIP primer comprises a primer sequence (F2) that is complementary to a sequence in the target nucleic acid molecule, and a tail sequence (F1c) that is not complementary to the sequence in the target nucleic acid molecule and that is 5′ of the primer sequence, and wherein the tail sequence is labeled with a quencher molecule; and
(ii) a detection probe (Fd) capable of hybridizing to the tail sequence of the FIP primer to form a duplex, wherein the detection probe is labeled with a fluorescent molecule that is quenched by the quencher molecule when the detection probe is hybridized to the tail sequence, and wherein more than 80% of the nucleotides in the detection probe are substituted in the 2′ position of its ribose ring with a methoxy group (OMe substituted);
(b) amplifying the target nucleic acid molecule by LAMP under suitable assay conditions that allow generation of the target amplicons by the LAMP reaction and multiple cycles of displacing the detection probe from the tail sequence, thereby releasing the quenching of the fluorescent molecule to generate a detectable signal; and
(c) detecting and optionally quantifying the signal from the detection probe, and therefrom determining the presence or amount of the target nucleic acid molecule in the sample.

2. The method of claim 1, wherein more than 90% of the nucleotides in the detection probe are OMe substituted.

3. The method of claim 2, wherein all of the nucleotides in the detection probe are OMe substituted.

4. The method of claim 1, wherein the DNA polymerase with strand displacement properties is Bst polymerase.

5. The method of claim 1, wherein the concentration of the detection probe is no more than 200 nM.

6. The method of claim 1, wherein the sample contains multiple target nucleic acid molecules.

7. The method of claim 6, wherein the LAMP reaction mixture is a multiplex reaction mixture capable of amplifying and detecting multiple target nucleic acid molecules.

8. The method of claim 7, wherein the multiplex reaction mixture comprises multiple detection probes, each probe being labeled with a different fluorescent molecule.

9. A method for detecting the presence of a target nucleic acid molecule in a sample using loop mediated isothermal amplification (LAMP), the method comprising:
(a) combining a LAMP reaction mixture, a DNA polymerase, and a sample containing the target nucleic acid molecule, wherein the LAMP reaction mixture comprises:
(i) a LAMP primer set, comprising a first inner primer (FIP), a second inner primer (BIP), a first outer primer (F3) and a second outer primer (B3), wherein FIP comprises a target complementary region (F2) and a tail sequence (F1c) 5′ of F2 and labeled with a quencher molecule, wherein F2 is complementary to a first region in the target nucleic acid molecule, and wherein F1c is identical to a second region in the target nucleic acid molecule 3′ of the first region, and
(ii) a detection probe (Fd) capable of hybridizing to F1c to form a duplex, wherein Fd is labeled with a fluorescent molecule that is quenched by the quencher molecule when Fd is hybridized to F1c, and wherein more than 80% of the nucleotides in Fd are substituted in the 2′ position of its ribose ring with a methoxy group;
(b) amplifying the target nucleic acid molecule by LAMP, thereby displacing Fd from F1c and generating a signal;
(c) detecting the signal from Fd; and
(d) determining from the signal the presence of the target nucleic acid molecule in the sample.

10. The method of claim 9, wherein the LAMP primer further comprises at least one of a first loop primer (Loop F) and a second loop primer (Loop B).

11. The method of claim 9, wherein more than 90% of the nucleotides in Fd are substituted in the 2′ position of its ribose ring with a methoxy group.

12. The method of claim 9, wherein all of the nucleotides in Fd are substituted in the 2′ position of its ribose ring with a methoxy group.

13. The method of claim 9, wherein the DNA polymerase is Bst polymerase.

14. The method of claim 9, wherein the concentration of Fd is no more than 200 nM.

15. The method of claim 9, wherein the LAMP reaction mixture is a multiplex reaction mixture capable of amplifying and detecting multiple target nucleic acid molecules.

16. The method of claim 15, wherein the multiplex reaction mixture comprises a plurality of detection probes, including Fd, each of the detection probes being labeled with a different fluorescent molecule.

17. A method of detecting a target nucleic acid molecule in a sample using detection of amplification by release of quenching loop mediated isothermal amplification (DARQ LAMP), the method comprising:
amplifying the target nucleic acid molecule by DARQ LAMP with a primer set comprising (i) a first inner primer (FIP), having a target complementary region (F2) and a tail sequence (F1c) 5′ of F2 and labeled with a quencher molecule, and (ii) a detection probe (Fd) capable of hybridizing to F1c to form a duplex, wherein Fd is labeled with a fluorescent molecule quenched by the quencher molecule when Fd is hybridized to F1c,
wherein at least 80% of the nucleotides in Fd are 2′-O-methylated.

18. The method of claim 17, wherein more than 90% of the nucleotides in Fd are 2′-O-methylated.

19. The method of claim 17, wherein all of the nucleotides in Fd are 2′-O-methylated.

20. The method of claim 17, wherein the concentration of Fd is no more than 200 nM.


SEQUENCE LISTING (35 sequences)

SEQ ID NO: 1 - DNA, 19 bp, synthetic construct
  misc_feature 1..19: note = SC2-N, F3 outer primer
  source 1..19: mol_type = other DNA
tggctactaccgaagagct

SEQ ID NO: 2 - DNA, 19 bp, synthetic construct
  misc_feature 1..19: note = SC2-N, B3 outer primer
  source 1..19: mol_type = other DNA
tgcagcattgttagcagga

SEQ ID NO: 3 - DNA, 41 bp, synthetic construct
  misc_feature 1..41: note = SC2-N, FIP inner primer
  misc_feature 1..41: note = 5` BHQ2 quencher
  source 1..41: mol_type = other DNA
tctggcccagttcctaggtagtccagacgaattcgtggtgg

SEQ ID NO: 4 - DNA, 40 bp, synthetic construct
  misc_feature 1..40: note = SC2-N, BIP inner primer
  source 1..40: mol_type = other DNA
agacggcatcatatgggttgcacgggtgccaatgtgatct

SEQ ID NO: 5 - DNA, 25 bp, synthetic construct
  misc_feature 1..25: note = SC2-N, Loop F loop primer
  source 1..25: mol_type = other DNA
ggactgagatctttcattttaccgt

SEQ ID NO: 6 - DNA, 21 bp, synthetic construct
  misc_feature 1..21: note = SC2-N, Loop B loop primer
  source 1..21: mol_type = other DNA
actgagggagccttgaataca

SEQ ID NO: 7 - DNA, 22 bp, synthetic construct
  misc_feature 1..22: note = SC2-N, Probe
  misc_feature 1..22: note = 3` Coumarin
  modified_base 1..22: mod_base = OTHER; note = 2'-O-methyl-ribose
  source 1..22: mol_type = other DNA
actacctaggaactgggccaga

SEQ ID NO: 8 - DNA, 18 bp, synthetic construct
  misc_feature 1..18: note = FluA H1, F3 outer primer
  source 1..18: mol_type = other DNA
agcaagaagttcaagccg

SEQ ID NO: 9 - DNA, 20 bp, synthetic construct
  misc_feature 1..20: note = FluA H1, B3 outer primer
  source 1..20: mol_type = other DNA
cgtgaactggtgtatctgaa

SEQ ID NO: 10 - DNA, 52 bp, synthetic construct
  misc_feature 1..52: note = FluA H1, FIP+4C inner primer
  misc_feature 1..52: note = 5` IBRQ Quencher
  source 1..52: mol_type = other DNA
ggctctactagtgtccagtaatagtccccaaatagcaataagacccaaagtg

SEQ ID NO: 11 - DNA, 47 bp, synthetic construct
  misc_feature 1..47: note = FluA H1, BIP inner primer
  source 1..47: mol_type = other DNA
ataacattcgaagcaactggaaatctgataataccagatccagcatt

SEQ ID NO: 12 - DNA, 17 bp, synthetic construct
  misc_feature 1..17: note = FluA H1, Loop F loop primer
  source 1..17: mol_type = other DNA
tctcccttcttgatccc

SEQ ID NO: 13 - DNA, 20 bp, synthetic construct
  misc_feature 1..20: note = FluA H1, Loop B loop primer
  source 1..20: mol_type = other DNA
tagtggtaccgagatatgca

SEQ ID NO: 14 - DNA, 29 bp, synthetic construct
  misc_feature 1..29: note = FluA H1, Probe+4G
  misc_feature 1..29: note = 3` FAM
  modified_base 1..29: mod_base = OTHER; note = 2`-O-methyl-ribose
  source 1..29: mol_type = other DNA
ggggactattactggacactagtagagcc

SEQ ID NO: 15 - DNA, 19 bp, synthetic construct
  misc_feature 1..19: note = FluA H3, F3 outer primer
  source 1..19: mol_type = other DNA
ggggttacttcaaaatacg

SEQ ID NO: 16 - DNA, 18 bp, synthetic construct
  misc_feature 1..18: note = FluA H3, B3 outer primer
  source 1..18: mol_type = other DNA
gttgccaatttcagagtg

SEQ ID NO: 17 - DNA, 51 bp, synthetic construct
  misc_feature 1..51: note = FluA H3, FIP+4C inner primer
  misc_feature 1..51: note = 5` IBRQ Quencher
  source 1..51: mol_type = other DNA
gagtgatgcattcagaattgcatttcccctgggaaaagctcaataatgaga

SEQ ID NO: 18 - DNA, 43 bp, synthetic construct
  misc_feature 1..43: note = FluA H3, BIP inner primer
  source 1..43: mol_type = other DNA
aatggaagcattcccaatgacagcttaacatatctgggacagg

SEQ ID NO: 19 - DNA, 17 bp, synthetic construct
  misc_feature 1..17: note = FluA H3, Loop F loop primer
  source 1..17: mol_type = other DNA
ccaatgggtgcatctga

SEQ ID NO: 20 - DNA, 20 bp, synthetic construct
  misc_feature 1..20: note = FluA H3, Loop B loop primer
  source 1..20: mol_type = other DNA
aaccattccaaaatgtaaac

SEQ ID NO: 21 - DNA, 29 bp, synthetic construct
  misc_feature 1..29: note = FluA H3, Probe+4G
  misc_feature 1..29: note = 3` HEX
  modified_base 1..29: mod_base = OTHER; note = 2`-O-methyl-ribose
  source 1..29: mol_type = other DNA
ggggaaatgcaattctgaatgcatcactc

SEQ ID NO: 22 - DNA, 25 bp, synthetic construct
  misc_feature 1..25: note = FluB NA, F3 outer primer
  source 1..25: mol_type = other DNA
caggaagagtaaaacatactgagga

SEQ ID NO: 23 - DNA, 18 bp, synthetic construct
  misc_feature 1..18: note = FluB NA, B3 outer primer
  source 1..18: mol_type = other DNA
gattcgcaaggccctgtt

SEQ ID NO: 24 - DNA, 48 bp, synthetic construct
  misc_feature 1..48: note = FluB NA, FIP+4C inner primer
  misc_feature 1..48: note = 5` IBRQ Quencher
  source 1..48: mol_type = other DNA
agggtctttttgctgtgtaactgttccccgcacatgcggatttgccag

SEQ ID NO: 25 - DNA, 42 bp, synthetic construct
  misc_feature 1..42: note = FluB NA, BIP inner primer
  source 1..42: mol_type = other DNA
gtggagactgatacagcggaatgcttccatcatttggtctgg

SEQ ID NO: 26 - DNA, 21 bp, synthetic construct
  misc_feature 1..21: note = FluB NA, Loop F loop primer
  source 1..21: mol_type = other DNA
ctacaggcacattctatggtt

SEQ ID NO: 27 - DNA, 18 bp, synthetic construct
  misc_feature 1..18: note = FluB NA, Loop B loop primer
  source 1..18: mol_type = other DNA
ataagattgatgtgcaca

SEQ ID NO: 28 - DNA, 29 bp, synthetic construct
  misc_feature 1..29: note = FluB NA, Probe+4G
  misc_feature 1..29: note = 3` JA270
  modified_base 1..29: mod_base = OTHER; note = 2`-O-methyl-ribose
  source 1..29: mol_type = other DNA
ggggaacagttacacagcaaaaagaccct

SEQ ID NO: 29 - DNA, 20 bp, synthetic construct
  misc_feature 1..20: note = GIC, F3 outer pirmer
  source 1..20: mol_type = other DNA
cgattttggttccggtcaca

SEQ ID NO: 30 - DNA, 19 bp, synthetic construct
  misc_feature 1..19: note = GIC, B3 outer pirmer
  source 1..19: mol_type = other DNA
cgggccgcttatacagtac

SEQ ID NO: 31 - DNA, 40 bp, synthetic construct
  misc_feature 1..40: note = GIC, FIP inner primer
  source 1..40: mol_type = other DNA
cgccccgactcacttaattgctccgcgccatacatgtcaa

SEQ ID NO: 32 - DNA, 40 bp, synthetic construct
  misc_feature 1..40: note = GIC, BIP inner primer
  misc_feature 1..40: note = 5` BHQ2 Quencher
  source 1..40: mol_type = other DNA
tgactcccacgtaaaaagcccccaatcgtcggttcgtgcg

SEQ ID NO: 33 - DNA, 22 bp, synthetic construct
  misc_feature 1..22: note = GIC, Loop F loop primer
  source 1..22: mol_type = other DNA
cgtcagttttctagcgttcgcc

SEQ ID NO: 34 - DNA, 24 bp, synthetic construct
  misc_feature 1..24: note = GIC, Loop B loop primer
  source 1..24: mol_type = other DNA
gcaccgttacgaagtatcaaaacg

SEQ ID NO: 35 - DNA, 22 bp, synthetic construct
  misc_feature 1..22: note = GIC, Probe
  misc_feature 1..22: note = 3` CY5.5
  modified_base 1..22: mod_base = OTHER; note = 2`-O-methyl-ribose
  source 1..22: mol_type = other DNA
ggggctttttacgtgggagtca